(19) United States
(12) Patent Application Publication
(10) Pub. No.: US 2016/0128868 A1
(43) Pub. Date: May 12, 2016

(54) EYE DROP CONTAINER WITH EYE DROP ALIGNMENT EFFECT

(71) Applicant: Hung-Lin LIN, New Taipei City (TW)

(72) Inventor: Hung-Lin LIN, New Taipei City (TW)

(21) Appl. No.: 14/537,465

(22) Filed: Nov. 10, 2014

(51) Int. Cl. A61F 9/00

(52) U.S. Cl. CPC A61F 9/0026

(57) ABSTRACT

An eye drop container having an eye drop alignment effect is provided. At the eye drop container, an alignment device that generates an alignment color light is provided. Thus, one is assisted to readily align an eye drop opening of the eye drop container to successfully drop eye drops in the eye drop container into one's lower eyelid. With the eye drop container of the present invention, a difficulty of aligning eye drops with one's lower eyelid is eliminated, and the eye drops can be controlled to be easily dropped into the lower eyelid, thereby providing an eye disease with an optimal treatment dosage.

### BACKGROUND OF THE INVENTION

a) Field of the Invention

The invention relates in general to an eye drop container with an eye drop alignment effect, and more particularly, to an alignment device disposed at a periphery of an eye drop container. Thus, a patient using the eye drop container is allowed to accurately drop eye drops into a lower eyelid according to an alignment colored light emitted from (or reflected by) the alignment device.

b) Description of the Prior Art

Mankind is the wisest of all creatures, and eyes are the windows to the soul. Almost every individual receives external messages through the eyes at all times. Particularly in the modern era with thriving technologies, as visual stimulations and vision utilization frequencies continue to increase, the number of patients with chronic or acute eye diseases is also ever-growing. Nonetheless, most patients are unaware of correct ways of using eye drops and thus fail to take best care of the eyes.

Referring to FIG. 1, when most people use eye drops (or referred to as an eye drop liquid), the eye drop liquid is usually directly applied by dropping it directly onto a surface of the eye E, in a way that a cornea of the eye E may become excessively irritated by such external stimulation, especially children may refuse to take the eye drop liquid due to intense discomforts. In addition to excessive irritation to the eye, the above inappropriate method of applying eye drops also exposes the eye to receiving eye drops with an unduly high concentration, such that the eye is prone to external damages to cause opposite and undesirable results.

Steps of a correct application procedure of eye drops recommended by a professional pharmacist are as follows. Step 1, shake an eye drop container to give an even concentration to eye drops in the container. Step 2, for a patient, tilt and lean the head backward and have the eye E look upward (looking toward the top of the head), as shown in FIG. 2. Step 3, pull a lower eyelid E1 downward by the index finger to reveal the conjunctival sac in the lower eyelid E1, and press a nasolacrimal duct between an inner side of the noise and the canthus of the eye by the middle finger. Step 4, hold the eye drop container with the other hand to suspend the eye drop container above the eye E and align the eye drop container with the lower eyelid E1. Step 5, squeeze the eye drop container to drop the eye drops into the lower eyelid E1. Step 6, lightly close the eye E and turn the eyeball several times to evenly apply the eye drops in the lower eyelid E1 to the surface (not shown) of the eye E.

Although being capable of preventing the conjunctiva of the eye E from excessive irritation, the steps of the above procedure for applying the eye drops are quite difficult to execute. One reason is that, whether external light is sufficient, it is rare that a patient accurately and successfully drop the eye drops into the lower eyelid. Instead, the patient may frequently drop the eye drops onto the face, as shown in FIG. 2. Apart from wasting the eye drops, the dosage of the eye drops applied cannot be easily controlled. As such, the effect of the treatment cannot be ensured and the eye is not given with the most appropriate care either.

Therefore, the present invention is directed to providing a solution for the predicament of dropping the eye drops all over the face and for enabling a patent to accurately apply the eye drops to a lower eyelid.

### SUMMARY OF THE INVENTION

In view of the above drawbacks, the primary object of the present invention is to provide an eye drop container for assisting a patient to accurately drop eye drops into a lower eye lid. To achieve the above object, the present invention provides an eye drop container having an eye drop alignment effect. The eye drop container includes a hollow bottle and a bottleneck portion. The bottleneck portion includes a front end having at least one eye drop opening, and a rear end that is connected to the hollow bottle as an integral to form a bottle shoulder. The present invention provides following features.

At least one alignment device is disposed in a surrounding manner at a middle section or the rear end of the bottleneck portion to keep the alignment device at an appropriate distance from the eye drop opening of the bottleneck portion. Thus, the patient is allowed to completely observe the alignment device with the eye to determine whether the eye drop opening aligns with the lower eyelid, thereby accurately dropping the eye drops into the lower eyelid and preventing waste of the eye drops caused by non-aligned positions when applying the eye drops.

According to the foregoing main feature, the alignment device is a loop accommodating body having a color different from those of the hollow bottle and the bottleneck portion. According to actual requirements, the loop accommodating body may be designed to actively or passively emit (or reflect) a mono-color or multi-color alignment color light. When the loop accommodating body is designed to passively emit (or reflect) an alignment color light, a surface of the loop accommodating body may be applied with or an internal of the loop accommodating body may be mixed with a mono-color or multi-color long-life fluorescent substance or a reflecting substance. When the loop accommodating body is designed to actively emit an alignment color light, a surface or an internal of the loop accommodating body is installed with a mono-color or multi-color active light emitting body and a light emitting body driving module for driving the light emitting body.

A main effect of the alignment color light defined in the present invention is to provide a distinguishing effect for the eye drop container, so as to allow a patient to easily identify a correct position of the alignment device.

The above and other aspects of the invention will become better understood with regard to the following detailed description of the preferred but non-limiting embodiments. The following description is made with reference to the accompanying drawings.

### BRIEF DESCRIPTION OF THE DRAWINGS

FIG. 1 is a schematic diagram of an incorrect procedure of directly applying conventional eye drops to an eye;

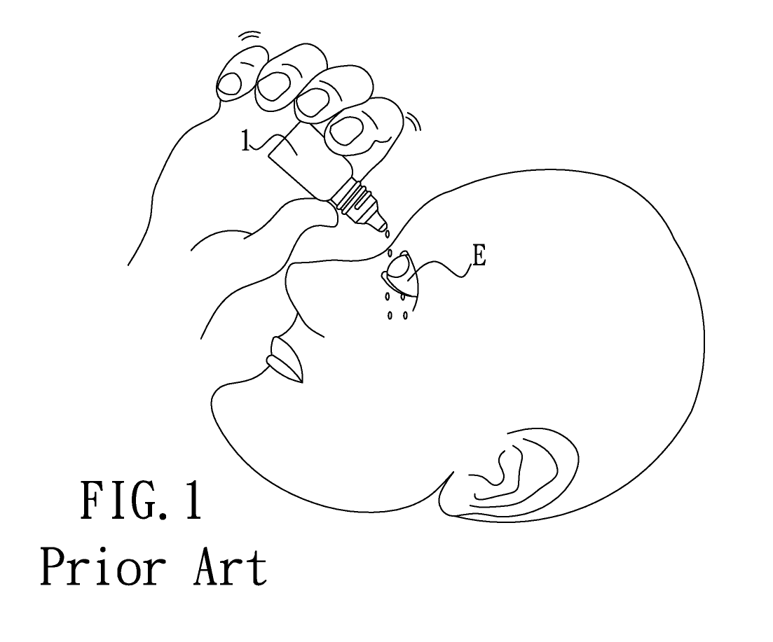

FIG. 2 is a front schematic diagram of non-aligned eye drop application positions during a correct procedure of utilizing a conventional eye drop container;

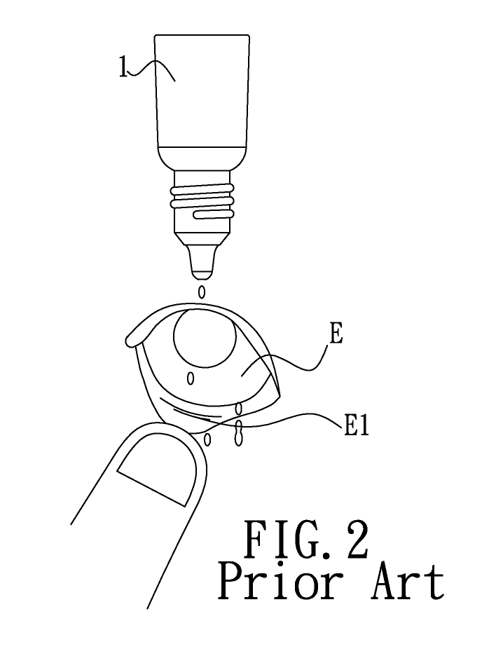

FIG. 3 is a side schematic diagram of an application state of an eye drop container having an eye drop alignment effect of the present invention;

FIG. 4 is a partial enlarged view of FIG. 3;

FIG. 5 is a front schematic diagram of an application state of an eye drop container having an eye drop alignment effect of the present invention; and

FIG. 6 is a front schematic diagram of another application state of an eye drop container having an eye drop alignment effect of the present invention, showing multiple active light emitting bodies disposed in a loop accommodating body.

### DETAILED DESCRIPTION OF THE PREFERRED EMBODIMENTS

The above and other aspects of the invention will become better understood with regard to the following detailed description of the preferred but non-limiting embodiments. The following description is made with reference to the accompanying drawings.

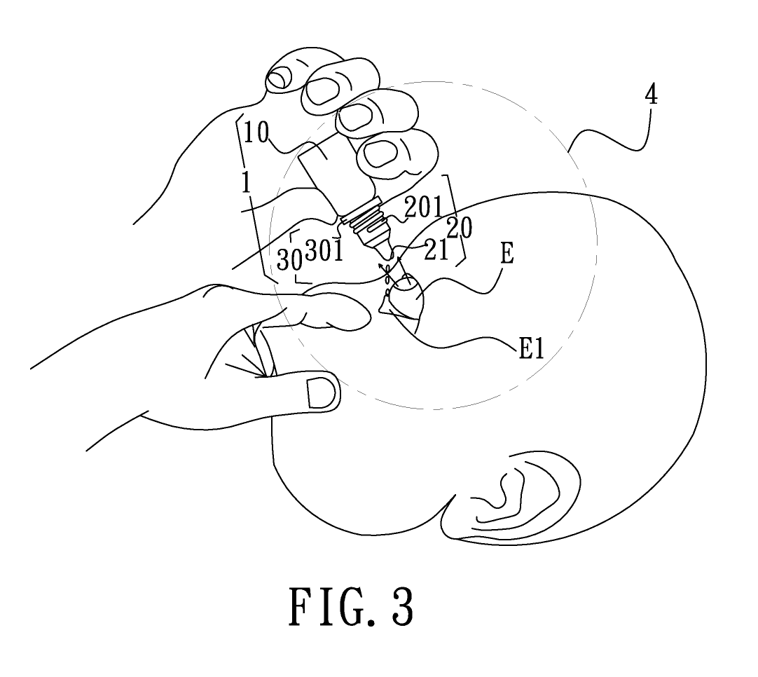

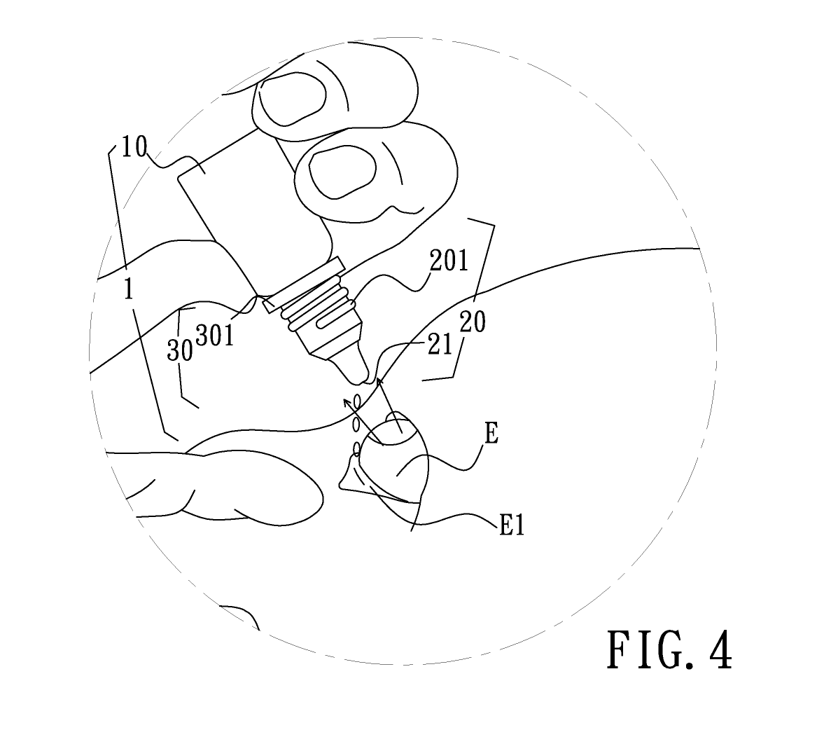

FIG. 3 shows a schematic diagram of an application state of an eye drop container having an eye drop alignment effect of the present invention. FIG. 4 shows a partial enlarged view of FIG. 3.

Referring to FIG. 3 and FIG. 4, an eye drop container 1 having an eye drop alignment effect provided by the present invention mainly includes a hollow bottle 10 adapted to be filled with eye drops and a bottleneck portion 20 having an eye drop opening 21. The bottleneck portion 20 and the hollow bottle 10 are combined to an integral.

The hollow bottle 10 serves a main purpose of accommodating eye drops, and has an appearance that is not limited to a circular, rectangular or polygonal body.

Preferably, the bottleneck portion 20 has a design as a hollow cylindrical body that is slenderer and longer than the hollow bottle 10. The bottleneck portion 20 has a middle section that may be provided with a screw thread 201 or a fastening structure (not shown) for coordinating with an external bottle cover, a front end provided with the eye drop opening 21, and a rear end connected to the hollow bottle 10 to form an integral bottle shoulder. Further, an internal part of the bottleneck portion 20 is in communication with the hollow bottle 10, such that the eye drops in the hollow bottle 10 is allowed to pass through the bottleneck portion 20 to be slowly released via the eye drop opening 21.

A novelty of the present invention is that, at least one alignment device 30 is provided in a surrounding manner at the middle section or the rear end of the bottleneck portion 20. Thus, the alignment device 30 is kept at an appropriate distance from the eye drop opening 21 of the bottleneck portion 20, so as to allow a patient to completely observe with the eye whether the eye drop opening 21 aligns with a lower eyelid E1, as shown in FIG. 3. Structure details of the alignment device 30 are not limited by the present invention. In the embodiment, the alignment device 30 is exemplified by a loop accommodating body 301 to illustrate effects of the alignment device 30 of the present invention. It should be noted that, the loop accommodating body 301 is an exemplary design for explaining the present invention and is not be construed as a limitation of the present invention.

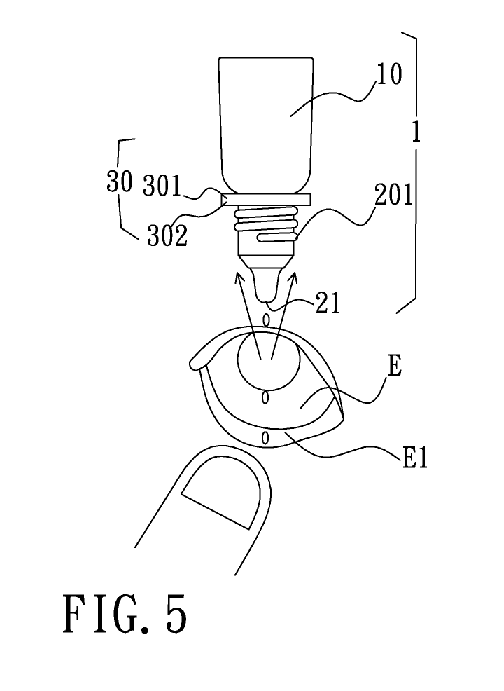

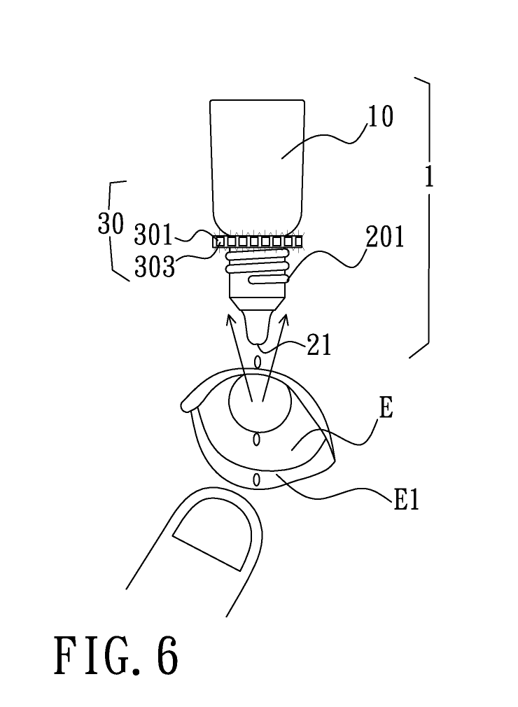

FIG. 5 is a front schematic diagram of an application state of an eye drop container having an eye drop alignment effect of the present invention. FIG. 6 is a front schematic diagram of another application state of an eye drop container having an eye drop alignment effect of the present invention, showing multiple active light emitting bodies disposed in a loop accommodating body.

Referring to FIG. 5 and FIG. 6, a main purpose of the alignment device 30 is to allow a patient to completely observe the loop accommodating body 301 (i.e., the alignment device 30) with an eye E to determine whether the eye drop opening 21 aligns with the lower eyelid E1. Hence, in the present invention, a color of the loop accommodating body 301 needs to be distinguished from that of the eye drop container 1, so as to allow the patient to easily identify a position of the loop accommodating body 301 and to prevent the patient from confusing the loop accommodating body 301 with the hollow bottle 10. Therefore, apart from designing a different color for an alignment color light generated by the loop accommodating body 301 from those of the hollow bottle 10 and the bottleneck portion 20, the loop accommodating body 301 may be further designed to actively or passively emit (or reflect) a mono-color or multi-color alignment color light. When the loop accommodating body 301 is designed to passively emit (or reflect) alignment color light, a surface of the loop accommodating body 301 may be applied by or an internal of the loop accommodating body 301 may be mixed with a mono-color or multi-color long-life fluorescent substance 302 or a reflecting substance, as shown in FIG. 5. When the loop accommodating body 301 is designed to actively emit an alignment color light, an internal of the loop accommodating body 301 is provided with a mono-color or multi-color active light emitting body 303 (e.g., a light-emitting diode (LED)), as shown in FIG. 6, and a light emitting body driving module (not shown) for driving the light emitting body. Details of circuit designs, a position of a switch or specifications of the type of battery (source) of the light emitting driving module, and a position of the of the light emitting driving module installed at the hollow bottle 10, are not the focus of the present invention and shall be omitted herein.

Steps of an application procedure of the present invention are similarly performed according to the foregoing recommended steps of a professional pharmacist. First of all, the hollow bottle 10 is shaken to give eye drops therein with an even concentration. The head of the patient is tilted and leaned backward to have the eye look upward (toward the top of the head). The lower eyelid E1 is pulled downward by the index finger to reveal the conjunctival sac under the lower eyelid (as shown in FIG. 3), the hollow bottle 10 of the present invention is held by the other hand, and the position of the hollow bottle 10 above the eye is adjusted until the eye completely observes the loop accommodating body 301 (as shown by the arrow in FIG. 4) at the bottleneck portion 20. As such, the eye drop opening 21 can be aligned with the position of the lower eyelid E1 of the eye E. The hollow bottle 10 is then squeezed to drop the eye drops via the eye drop opening 21 into the lower eyelid E1. The eye is closed lightly and the eyeball is turned several times to evenly apply the solution in the lower eyelid E1 (not shown) to complete the eye drop application procedure of the present invention.

While the invention has been described by way of example and in terms of the preferred embodiments, it is to be understood that the invention is not limited thereto. For one person skilled in the art, it is to be understood that the non-limiting embodiments are intended to cover various modifications and similar arrangements and procedures based on the disclosed technical contents, and the scope of the appended claims therefore should be accorded the broadest interpretation so as to encompass all such modifications and similar arrangements and procedures based on the disclosed technical contents.

What is claimed is:

1. An eye drop container having an eye drop alignment effect, the eye drop container comprising a hollow bottle and a bottleneck portion having an eye drop opening, the bottleneck portion being connected to the hollow bottle to form a bottle shoulder; the eye drop container having:
at least one alignment device disposed in a surrounding manner at a middle section or a rear end of the bottleneck portion, and the alignment device is kept at an appropriate distance from the eye drop opening of the bottleneck portion, such that one is able to completely observe the alignment device with an eye to determine whether the eye drop opening aligns with one's lower eyelid.

2. The eye drop container having an eye drop alignment effect according to claim 1, wherein the alignment device is a loop accommodating body.

3. The eye drop container having an eye drop alignment effect according to claim 1, wherein a surface or an internal of the alignment device is provided with a mono-color or multi-color long-life fluorescent substance for generating an alignment color light.

4. The eye drop container having an eye drop alignment effect according to claim 1, wherein a surface or an internal of the alignment device is provided with a mono-color or multi-color active light emitting body for generating an alignment color light.

5. The eye drop container having an eye drop alignment effect according to claim 4, wherein the internal of the alignment device is provided with a light emitting body driving module for driving the active light emitting body to generate the alignment color light.
